(19) United States
(12) Patent Application Publication
(10) Pub. No.: US 2013/0288376 A1
(43) Pub. Date: Oct. 31, 2013

(54) SYSTEM FOR AND METHOD OF SEPARATING GERM FROM GRAINS USED FOR ALCOHOL PRODUCTION

(71) Applicant: Chie Ying Lee, Fremont, CA (US)

(72) Inventor: Chie Ying Lee, Fremont, CA (US)

(21) Appl. No.: 13/864,751

(22) Filed: Apr. 17, 2013

Related U.S. Application Data
(60) Provisional application No. 61/638,455, filed on Apr. 25, 2012.

(51) Int. Cl. C12N 5/04; C07K 14/415; C11B 1/10

(52) U.S. Cl. CPC C12N 5/04; C11B 1/10; C07K 14/415
USPC 435/410; 554/9; 435/294.1; 530/372

(57) ABSTRACT

Methods of and device for separating germs from grains used for alcohol production are provided. The principle of density difference is able to be used to separate germs from a slurry. By adjusting the density and/or viscosity of the slurry, higher germ recovering rate is attended. The method of adjusting the density and/or viscosity includes recycling a cook-water to reduce the viscosity of the slurry before fermentation and pre-concentrating a whole stillage at an evaporator and adding a syrup to the whole stillage after fermentation. An air flotation unit is able to be used to increase oil and germ recovery rate.

### CROSS REFERENCE TO RELATED APPLICATIONS

This application claims priority from U.S. Provisional Patent Application Ser. No. 61/638,455, filed Apr. 25, 2012 and entitled “A SYSTEM FOR AND METHOD OF SEPARATING GERM FROM GRAINS USED FOR ALCOHOL PRODUCTION,” which is hereby incorporated herein by reference in its entirety for all purposes.

### FIELD OF THE INVENTION

The present invention relates to a system for and method of separating by-products including oil, germ, and protein from grains that are used for alcohol production.

### BACKGROUND

FIG. 1 is a flow diagram of a typical wet milling ethanol production process. FIG. 2 is a flow diagram of a typical wet milling ethanol production process. FIG. 3 is a flow diagram of a dry fraction ethanol production process.

Ethanol is gaining great interest today. Ethanol can be produced from virtually any type of grains, but it is mostly often made from corns. Most of the fuel ethanol in the United States is produced from a wet milling process or a dry grinding ethanol process. Although virtually any type and quality of grains can be used to produce ethanol, the feedstock for these processes is typically a corn known as “No. 2 Yellow Dent Corn.” The “No. 2” refers to a quality of corn having certain characteristics as defined by the National Grain Inspection Association, as is known in the art. “Yellow Dent” refers to a specific type of corn as is known in the art.

The conventional methods of producing various types of alcohols from grains generally follow similar procedures, depending on whether the process is operated wet or dry. Wet milling corn processing plants convert corn grain into several different co-products, such as germ (for oil extraction), gluten feed (high fiber animal feed), gluten meal (high protein animal feed), and starch-based products (such as ethanol, high fructose corn syrup, or food and industrial starch). Dry grinding ethanol plants convert corn into two products, namely ethanol and distiller's grains with soluble. If the distiller's wet grains with soluble is sold as wet animal feed, it is referred to as DWGS. If the distiller's wet grains with soluble is dried for animal feed, it is referred to as DDGS. In a standard dry grinding ethanol process, one bushel of corn yields approximately 8.2 kg (approximately 17 lbs) of DDGS in addition to the approximately 10.3 liters (approximately 2.75 gal) of ethanol. The DDGS, a co-product of the ethanol, provides a critical secondary revenue stream that offsets a portion of the overall ethanol production cost.

With respect to the wet milling process, FIG. 1 is a flow diagram of a typical wet milling ethanol production process 20. The process 20 begins with a steeping Step 21. At the steeping Step 21, corn is soaked for 24 to 48 hours in a solution of water and sulfur dioxide in order to soften the kernels for easier grinding, leaching soluble components into the steep water, and loosening the protein matrix with the endosperm. Corn kernels contain mainly starch, fiber, protein, and oil. At a milling Step 22, a mixture of steeped corn and water is then fed to a degerminating mill, in which the corn is ground in a manner that the kernels are torn open and the germs are released, such that a heavy density (8 to 9.5 Be) slurry of the ground components is made, which contains primarily a starch slurry.

At a germ separating Step 23, a hydro-cyclone(s) is used to separate the germs from the rest of the slurry by floating the germs on top of the slurry. The germs are part of the kernels that contain oil of the corns. The separated germ stream at the germ separating Step 23 (containing some portion of the starch, protein, and fiber) is sent to a germ washing process to remove starch and protein and then sent to a dryer to produce about 2.5 to 3 Lb. (dry basis) of germ per bushel of corn. The dry germs have about 50% oil content on a dry basis.

The remaining slurry (now devoid of germ, but contains fiber, gluten (e.g., protein), and starch) of the germ separating Step 23 is then subjected to a fine grinding Step 24 (second grinding). The fine grinding Step 24 releases endosperm components, such as gluten and starch, from the fiber. At a fiber separating Step 25, the slurry from the fine grinding Step 24 passes through a series of screens in order to separate the fiber from starch and gluten and wash the fiber so that the fiber is free of gluten and starch. The fiber separating Step 25 typically uses static pressure screens or rotating paddles mounted in a cylindrical screen (Paddle Screens).

Even after washing, the fiber from a typical wet grinding mill still contains 15 to 20% starch. Normally, this starch is sold with the fiber as animal feed. The remaining slurry, which is now devoid of fiber, is subjected to a gluten separating Step 26. At the gluten separating Step 26, centrifuges or hydro-cyclones are used to separate starch from the gluten. The gluten stream of the gluten separating Step 26 is sent to a vacuum filtering Step 31 and a dryer to produce gluten (protein) meal. The starch stream of the gluten separating Step 26 is sent to a starch purifying Step 27, where the starch is purified. The resulting purified starch co-product is sent to a jet cooking process at a liquefying and saccharifying Step 28 to start the process of converting the starch to sugar. Jet cooking refers to a cooking process performed at elevated temperatures and pressures. Typically, jet cooking occurs at a temperature of about 120° C. to 150° C. (about 248 to 302° F.) and a pressure of about 8.4 to 10.5 kg/cm² (about 120 to 150 lbs/in²), although the temperature can be as low as about 104° C. to 107° C. (about 220° F. to 225° F.) when pressures of about 8.4 kg/cm² (about 120 lbs/in²) are used.

The liquefying and saccharifying Step 28 comprises the process of liquefying and saccharifying. The process of liquefying comprises holding a solution at 90° C. to 95° C. for an alpha-amylase enzyme hydrolyzing the gelatinized starch into maltodextrins and oligosaccharides (chained glucose sugar molecules), such that a liquefied mash or slurry is produced. The process of saccharifying at the Step 28 contains cooling the liquefied mash from the liquefying process to about 50° C. and adding a commercial available enzyme, which is known as gluco-amylase. The gluco-amylase hydrolyzes the maltodextrins and short-chained oligosaccharides into single glucose sugar molecules to produce a liquefied mash. In a fermenting Step 29, a common strain of yeast (Saccharomyces cerevisae) is added to metabolize the glucose sugars into ethanol and CO₂.

Upon completion of the liquefying and saccharifying Step 28, the fermented mash (“beer”) contains about 17% to 18% ethanol (volume/volume basis) with soluble and insoluble solids from all of the remaining grain components. The remaining solids and some liquid after the liquefying and saccharifying Step 28 are sent to an evaporator for an evaporating Step 30, where yeast is recovered as a byproduct. The recycled yeast can be optionally recycled back to the fermenter at the fermenting and distilling Step 29. In some instances, the CO₂ is recovered and sold as a commodity product.

At the fermenting and distilling (dehydrating) Step 29, the beer is pumped into distilling columns and boiled to vaporize the ethanol. The ethanol vapor is condensed in the distillation columns. The condensed ethanol becomes liquid alcohol (in this instance, ethanol) exits the top of the distillation columns at about 95% purity (190 proof). The 190 proof ethanol then goes through a molecular sieve dehydrating column, which removes the remaining residual water from the ethanol to generate a final product of essentially 100% ethanol (199.5 proof). This anhydrous ethanol is now ready to be used for motor fuel purposes.

No centrifugation step is needed at the end of the wet milling ethanol production process 20 as the germ, fiber and gluten have already been removed in the previous separation Steps 23, 25, and 26. The “stillage” produced after distillation and dehydration 29 in the wet milling process 20 comprises a “syrup.”

The wet grinding process 20 can produce a high quality starch product and separated germ, fiber and protein. The high quality starch product can be used to be converted to alcohol. Germs, fiber and proteins can be sold as by-products to generate additional revenue streams. However, the overall yields for various by-products are less than desirable and the wet grinding process is complicated and costly requiring high capital investment as well as high-energy costs for operation.

Because the capital cost of wet grinding mills can be so prohibitive, some alcohol plants prefer to use a simpler dry grinding process. FIG. 2 is a flow diagram of a typical dry grinding ethanol production process 10. As a general reference point, the dry grinding ethanol process 10 can be divided into a front-end part and a back-end part. The part of the process 10 that occurs prior to distillation and dehydration 14 is considered the “front-end”, and the part of the process 10 that occurs after distillation and dehydration 14 is considered the “back-end.”

The front-end of the process 10 begins with a grinding Step 11. At the grinding Step 11, dried whole corn kernels are passed through hammer mills for grinding into meal or a fine powder. The screen openings in the hammer mills are typically of a size #7 or about 2.78 mm. The screen openings result in a very wide spread particle distribution curve having particle sizes as small as 45 micron and as large as 2 to 3 mm. Next, the ground meal is mixed with water to create slurry, and a commercially available enzyme called alpha-amylase is added (not shown). This slurry is then heated to approximately 120° C. for about 0.5 to 3 minutes in a pressurized jet cooker at a liquefying Step 12 in order to gelatinize (solubilize) the starch in the ground meal. It is noted that some processes are not equipped with a jet cooker and use a longer holding time instead.

At a liquefying Step 12, a ground meal is mixed with cook water to create slurry and a commercially available enzyme called alpha-amylase is typically added (not shown). The pH is adjusted here to about 5.8 to 6 and the temperature is maintained between 50° C. to 105° C., such that the insoluble starch in the slurry is able to be converted to a soluble starch. The stream after the liquefaction Step 12 has about 30% dry solids (DS) content with all the components contained in the corn kernels including sugars, protein, fiber, starch, germ, grit, oil and salts. There are generally three types of solids in the liquefied stream: fiber, germ, and grit. The particle size distribution of all three solids are about the same. The liquefying Step 12 is followed by a simultaneous saccharifying and fermenting Step 13. These simultaneous steps are referred to in the industry as “Simultaneous Saccharification and Fermentation” (SSF). In some commercial dry grind ethanol processes, saccharification and fermentation occur separately (not shown). Both individual saccharification and SSF can take as long as about 50 to 60 hours. Fermentation converts the sugar to alcohol using a fermenter. At a distilling (and dehydrating) Step 14, a still is used to recover the alcohol.

The back-end of the process 10 (at and after the distilling Step 14) includes a centrifuging Step 15, which involves centrifuging the residuals (i.e., “whole stillage”, produced at the distilling Step 14) to separate the insoluble solids (“wet cake”) from the liquid (“thin stillage”). The “wet cake” includes three types of fibers including: (1) pericarp (typically having average particle sizes about 1 mm to 3 mm); (2) tricap (having an average particle size about 500 micron); and (3) fine fiber (having an average particle size about 150 micron). The liquid from the centrifuge contains about 6% to 8% DS, which contains mainly oil, protein, soluble solid from fermenter and ash from corns.

At an evaporating Step 17, the thin stillage enters evaporators to boil away moisture leaving a thick syrup that contains the soluble (dissolved) solids from fermentation (25% to 40% dry solids). The concentrated slurry can be subjected to an optional oil recovery Step 16. At the oil recovering Step 16, the slurry can be centrifuged to separate oil from the syrup. The oil can be sold as a separate high value product. The oil yield is normally about 0.4 lb./Bu of corn with high free fatty acids content. This oil yield shows only about ¼ of the oil in the corn are recovered. About one-half of the oil inside the corn kernel still remains inside the germ after the distilling Step 14, which cannot be separated in a typical dry grind process using centrifuges. The free fatty acids content (created when the oil is held in the fermenter for approximately 50 hours) reduces the value of the oil. The (de-oil) centrifuge only removes less than 50% because the protein and oil make emulsions, which cannot be satisfactorily separated.

The centrifuged wet cake and the syrup (containing more than 10% oil) can be mixed together and the mixture can be sold to beef and dairy feedlots as Distillers Wet Grain with Soluble (DWGS). Alternatively, the syrup can be mixed with the wet cake, and the concentrated syrup mixture can be dried in a dryer and sold as Distillers Dried Grain with Soluble (DDGS) to dairy and beef feedlots. This DDGS has all the protein and 75% of the oil in corn. However, the value of DDGS is low due to the high percentage of fiber and the oil. The oil sometimes makes the animals hard to digest the DDGS.

Because the dry mill process 10 only produces ethanol and low value DDGS, many companies have started to develop a dry fraction process, which is illustrated in FIG. 3. In the dry fraction process 30, corn goes through a pretreatment Step 31, such as a steam treatment. At a dry fractioning Step 31, various types of mechanical separation equipments are utilized to separate the dry fractions of the corn including fiber, starch, and oil/germ portion. While this dry fractioning Step 32 accomplishes some separation of the components, the separation is generally incomplete. For example, the fiber portion normally contains more than 30% starch on a dry basis and the germ contains more than 25% starch and 35% oil on a dry basis. Only less than 30% of the total oil in the corn kernels is recovered with these processes. The germ and fiber portions must go through further purification stages before they can be sold for a reasonable price.

After the dry fractioning Step 32, the starch (with protein) goes through another grinding step, a liquefying Step 33, a fermenting Step 34, a distilling Step 35, and evaporating Step 36 to produce alcohol and syrup. The above procedure is similar to the procedure described in the dry grinding process 10. However, the alcohol yield using the dry fraction process is normally lower (e.g., 2.3 gal/Bu of corn), because starch is mixed with the germ and fiber portions. In addition, the purification steps mentioned above for the germ and fiber are complicated and costly. Notably, the dry fractioning process does not give sharp separation and produces low purity by-products, which complicates the downstream purification steps. Because of the high costs and low yields, these dry fractionation processes have not been generally accepted by the industry.

Other attempts have been made in the dry grinding industry to recover high value by-products, such as oil. However, attempts to separate oil from the “hammer milled” slurry have failed because of the high concentration of solids and because the oil is not released from the solid particles. Some success has been realized with processes recovering oil at the evaporating steps of the dry mill process. However, the yield is relatively low, and the oil must move through the entire process, including fermentation, prior to evaporation. The presence of oil in these steps of the process can be detrimental to the efficiency of the remaining parts of the process. Attempts have been made to recover the oil directly after fermentation. However, the process of mixing and fermenting emulsifies the oil. The oil in a form of emulsion makes it very difficult to remove. Costly chemicals (emulsion breaker) are needed to give reasonable oil yield. Normal 0.4 lb./Bu of oil can be obtained without emulsion breaker and 0.6 lb./Bu of oil can be obtained with emulsion breaker. Other attempts have been made to recover oil directly from corn by using solvent extraction. However, the cost is too high for commercial uses.

In one of the patent applications (U.S. patent application Ser. No. 13/428,263, titled “DRY GRIND ETHANOL PRODUCTION PROCESS AND SYSTEM WITH FRONT END MILLING METHOD,” filed on Mar. 23, 2012), multi stages of a dewatered grinding mill is used to grind the mix germ/fiber particles. The patent application mentioned above is incorporated by reference in its entirety for all purposes. Both germ/fiber particles described in the patent application have wide range of particle size distribution, from less than 45 micron to as large or larger than 1,500 micron. Softening the germ particle in a lower Brix solution at longer holding tank can make germ much softer and easy to break up than fiber. Accordingly, the dewatered milling method can break up more germ particle than fiber. However, each dewatered milling step only can reduce the germ particle size about half of size at best. For example, the germ particle size of average of 1000 micron germ particle size will become about average of 600 micron size after one pass of dewatered milling step. The ideal germ particle size to release oil is preferred to be less than 150 micron size. Therefore, normally at least two/three stage dewatered milling in series are needed to release more oil from the germ particles. The dewatered milling equipment costs plus the power consumption on dewatered milling step prevent the industry from adding more dewatered milling steps. In addition, if the corn that is used is old or dried in a high temperature environment, the germ particle softening process becomes very slow during the holding tank softening process. In such case, more enzyme and larger holding tank (to give longer holding time to soften germ) are often used to give a desired oil yield.

The other possible approach involves separating germs by using heavy density media methods (U.S. patent application Ser. No. 13/377,353, filed on Dec. 9, 2011, which is incorporated by reference in its entirety for all purposes). Germs are separated from fiber by their density difference between germ (1 to 1.05 gram/ml) and fiber (1.1 to 1.2 gram/ml). The density of liquefied starch solution at the liquefying step is about 1.1 to 1.13 (about 25 to 30% DS). Accordingly, the germ particles float on this liquefied starch solution (a heavy density liquid media) and the fiber particles sink in this heavy density liquid media. Nonetheless, this liquefied starch solution has a very high viscosity; it makes this germ/fiber separation much harder. The normal hydro-cyclone is not appropriate to be used on this type of heavy density liquid media due to the high viscosity of the liquefied starch. The three phase decanter has success in separating oil (as a light phase), germ in liquefied starch solution (as a heavy phase), and fiber (as a solid phase) from liquefied slurry right after jet cooker. About 0.5 lb./Bu oil and about 2 lb./Bu germ are separated from liquefied starch slurry. The equipment cost and operation cost prevents from proceeding further.

There are about 5.1 lb./Bu of germs in corn kernel and about 1.9 lb./Bu oil in corn kernel (about 1.5 lb./Bu oil in the germ and 0.3 lb./Bu oil in endosperm). In the typical dry mill process, about 0.5 to 1 lb./Bu of oil is released out and about 0.9 to 1.2 lb./Bu of oil remains inside the germ. The whole stillage has about 2 to 3 lb./Bu of germs and 4 to 5 lb./Bu fiber. Both solids have the same particle size ranges from less than 50 micron up to more than 1 mm. The whole stillage normally has 13 to 14% DS (density of about 1.05 gram/ml). Normally, decanter is used to separate decanter cake (DDG with 35% DS) from the thin stillage (with 8% DS and density of 1.03 gram/ml) that are contained in the whole stillage. The majority of germ is trapped by fiber and discharged with fiber as decanter cake (DDG). Only a small portion (around 10 to 30% germs) of small germ particles (the particle size smaller than 300 micron) is carried with fine fiber/protein solution as decanter over flow (thin stillage). The fine germ has about the same particle size range as fine fibers (smaller than 45 micron up to 300 micron). It is not easy to separate the fine germ particles from fine fiber in a protein solution.

### SUMMARY OF THE INVENTION

The heavy density media separation method cannot be easily applied on the typical dry mill processes, because the whole stillage in the back-end (after a fermenting step) has a density that is too low and the liquefied starch solution in the front-end has a viscosity/density that is too high. The present invention is able to adjust density/viscosity of slurry on the dry process either in the back-end or front-end, such that the heavy density media separation method (heavy density liquid media classification principle) can be effectively applied to a dry milling process to separate the germ particles from fibers. As a result, more oil is able to be extracted and recovered from the separated germs in a dry mill process. Any other methods that are able to be used to adjust the density/viscosity of slurry to facilitate the germ separation are within the scope of the present invention, such as adding supercritical CO₂ to adjust the density of the slurry.

A method of and device for using heavy density liquid media principle to separate the germ from fiber on a liquefaction stage is provided. Details of a related process is described in the U.S. patent application Ser. No. 13/377,353, titled “A SYSTEM AND METHOD FOR SEPARATING HIGH VALUE BY-PRODUCTS FROM GRAINS USED FOR ALCOHOL PRODUCTION,” which is incorporated by reference in its entirety for all purposes. In the related process, the oil and germ can be separated from fiber during the liquefying stage by using a three phase decanter. In another related process, a multi-stage dewatering grinding mill is used to grind the mix germ/fiber particle, which is described in the U.S. patent application Ser. No. 13/428,263, titled “DRY GRIND ETHANOL PRODUCTION PROCESS AND SYSTEM WITH FRONT END MILLING METHOD,” filed on Mar. 23, 2012, which is incorporated by reference in its entirety for all purposes.

The above mentioned systems and methods are able to be combined with the embodiments of the present invention. In some embodiments of the present invention, the Brix at the two holding tanks can be adjusted by adding an amount of back-set solution/fluid to one or both of the holding tanks as shown in the flow sheets in FIG. 4 to FIG. 7B. As show in the FIG. 4, the density of the slurry in the two holding tanks can be adjusted by the amount of back-set solution added to one or both of the holding tanks. For example, the slurry with a desired density from 6 to 24 Brix in one case and 10 to 17 Brix (density of 1.05 to 1.1 gram/ml) in the other case are able to be obtained. A person of ordinary skill in the art appreciates that any selected Brix and density are within the scope of the present invention so long as the adjusted slurry can facilitate the separation or have a better separation of germ. Next, the slurry is sent to a one or multi-stage germ separation device to separate the germs from the fibers, which is a process similar to the procedure of the corn wet milling process.

The principle of separation by density difference is based on that the germ and the fibers have different densities in a heavy density liquid, so the germs and fibers behave differently in the heavy density liquid. The germ is lighter than the heavy density liquid (higher density) (1.05 to 1.1 gram/ml), so the germ floats to the top portion/layer of the slurry. Fibers are heavier than the heavy density liquid, so the fibers sink to the bottom portion/layer of the slurry. By using the separation methods described above, better separation between germ and fiber are able to be obtained.

The above heavy density media embodiment can also be applied to the back-end of the alcohol production process. However, the whole stillage at the back-end has only about 13% to 15% DS (density of 1.05 gram/ml) and soluble solid is only about 3 to 5% DS. The thin stillage contains 6 to 8% DS (density of around 1.03 gram/ml), which is not heavy enough as a floating solution to float the germ in the dry milling process. The typical dry milling process uses a decanter to separate the whole stillage. The decanter cake (DDG) contains more than 70% of germ in the whole stillage, only about 10 to 30% of germ in the whole stillage are separated out in the decanter overflow (thin stillage). The thin stillage contains soluble solid, protein (less than 50 micron), small germ particle and fine fiber. Both germ and fiber have about the same particle size range (less than 50 micron to about 200 micron). So when a screen is used to recover the germ, the germ also carries fine fiber. This germ/fine fiber mixture creates the problem downstream in the oil extraction process.

The germ that is recovered from the whole stillage can be further improved by increasing the density of liquid in the whole stillage, so the heavy density media embodiments described above can be applied to give better separation between the fibers and germ by the differences of their density.

There are two ways to increase the liquid density in the whole stillage including a) sending the whole stillage to the first stage evaporator to increase the slurry solid content having 17 to 24% DS and b) adding concentrated syrup back to the whole stillage to form a 17 to 24% DS slurry. As shown in FIG. 5, FIG. 5A and FIG. 7A, the whole stillage is sent to the first stage evaporator to be pre-concentrated to 17˜24% DS to increase the density of the slurry. The heavy density slurry is then sent to a germ separator to separate germs from the fibers by the differences in their densities as described above. As shown in the FIG. 6, FIG. 6A, and FIG. 7B, the portion of the heavy syrup can be recycled back to be mixed with the whole stillage to increase the density of the slurry, then the syrup is sent to the germ cyclone to float the germ and to be separated from the fibers.

The separated germ can be sent back to the front-end dewatered milling system or be sent to a separated small fine grinding mill to grind all the germ particles to be smaller than 150 micron to release more oil. Cell breaking type of enzymes are able to be added before, during and/or after this fine germ grinding step to help release more oil. The pH can be adjusted to the range of 7 to 9 by using weak Na₂CO₃ before, during, and/or after the fine grinding step. The basic environment provided by the weak Na₂CO₃ can help release more oil from the fine grinded germ particles. The fine grinded germ slurry can be sent to the front-end or the back-end oil recovery system, and/or followed by 200 proof alcohol extraction Step 117.

As shown in the FIG. 7, FIG. 7A, and FIG. 7B, the air floatation unit is added to the current thin stillage tank to create fine air bubbles. The fine air bubbles float the oil/fine germ particle/protein out to be on top of the thin stillage tank. The top layer (oil/emulsion) is sent back to the front-end as part of the back-set solution for more oil recovery. The increased density of the thin stillage by pre-concentrating the slurry or by adding the recycled syrup can increase the air floatation unit efficiency as well. The addition of the protein separation step at the bottom of the thin stillage tank to remove protein and break the bonds between the oil/protein also helps the air floatation unit. If Na₂CO₃ is used for pretreating the fine ground germ, the addition of this pretreated slurry with Na₂CO₃ fine ground germ slurry to the thin stillage can form very fine CO₂ bubbles, which also increase the air floatation unit efficiency.

In the following, some aspects of the present invention are provided. In an aspect, a method of separating germs from a whole stillage comprises increasing a liquid density of a higher density layer of a slurry after a distilling/fermenting step. In some embodiments, the increasing the liquid density comprises making the higher density layer having a solid content in the range of 15%-30% DS. In other embodiments, the increasing the liquid density comprises optimizing the higher density layer having a solid content in the range of 17%-24% DS. In some other embodiments, the increasing the liquid density comprises sending the whole stillage to an evaporator before separating germs from a solution. In some embodiments, the increasing the liquid density comprises adding a concentrated syrup to the whole stillage. In other embodiments, the slurry comprises the whole stillage. In some other embodiments, the method further comprises separating germs from fiber using a germ cyclone, a decanter or a combination thereof. In some other embodiments, the germ cyclone, the decanter or both comprises a single or a multiple stage separating device. In some embodiments, the germ cyclone, the decanter, or both comprises a counter current setup. In other embodiments, the method further comprises recovering germ from a thin stillage layer. In some other embodiments, the method further comprises releasing oil by dewater milling the germ. In some embodiments, the method further comprises breaking a germ cell wall by the dewater milling. In other embodiments, the method further comprises breaking a germ cell wall by adding a cell breaking enzyme. In some other embodiments, the method further comprises adjusting a pH value to be in the range of 6 to 9 before dewater milling. In some embodiments, the method further comprises recycling a portion of a de-germ thin stillage solution to dilute a feed to a germ cyclone such that a germ and fiber separating efficiency is improved. In some other embodiments, the method further comprises floating fine germ particles, oil emulsion, or both by an air flotation system.

In another aspect, an alcohol production system comprises a first germ cyclone, a first decanter, or a combination thereof after a distiller/fermenter configured to separate germs from fiber. In some embodiments, the system further comprises a second germ cyclone, a second decanter, or a combination thereof before the fermenter. In other embodiments, the system further comprises an evaporator before the first germ cyclone, the first decanter, or the combination thereof. In some embodiments, the system further comprises a distillation device before the first germ cyclone, the first decanter, or the combination thereof. In some other embodiments, the system further comprises a dewater milling device before the fermenter. In some embodiments, the system further comprises a protein recovery device. In other embodiments, the system further comprises an oil recovering device before the fermenter. In some other embodiments, the system further comprises an air floating device for recovering the germs in a thin stillage.

In another aspect, a method of recovering germs comprises reducing a viscosity of a liquefied starch solution. In some embodiments, the liquefied starch solution has a reduced viscosity in the range of 12% and 23% Brix. In other embodiments, the liquefied starch solution has a density in the range of 1.01 to 1.125. In some other embodiments, the reducing the viscosity is done by adding cook-water. In some other embodiments, the cook-water comprises a counter current after separating germs from fiber. In some embodiments, the reducing the viscosity occurs before or at separating germs from fiber.

In another aspect, a method of recovering germs from a liquefied starch solution comprising separating germs from fiber before fermenting using a germ cyclone, a decanter, or a combination thereof. In some embodiments, the method further comprises recovering the germs from the liquefied starch solution. In other embodiments, the method further comprises breaking cell walls of the germs to release oil by dewater milling. In some other embodiments, the method further comprises adjusting a pH value in a range of 6 to 9 before the dewater milling. In some embodiments, the method further comprises breaking cell walls of the germs by adding an enzyme. In other embodiments, the method further comprises recycling a portion of a de-germ liquefied starch solution to dilute a feed to the germ cyclone, the decanter, or the combination thereof. In some other embodiments, the germ cyclone, the decanter, or the combination thereof comprises a single stage setup. In some embodiments, the germ cyclone, the decanter, or the combination thereof comprises a multiple stage setup. In other embodiments, the germ cyclone, the decanter, or the combination thereof comprises a counter current setup. In some other embodiments, the liquefied starch solution has a viscosity in a range of 8 to 35 Brix. In some embodiments, the method further comprises adjusting a viscosity of the liquefied starch solution by using a counter current washing process. In some other embodiments, the method further comprises dewater milling before the fermenting. In some embodiments, the method further comprises recovering protein. In other embodiments, the method further comprises recovering oil before the fermenting.

### BRIEF DESCRIPTION OF THE DRAWINGS

The accompanying drawings, which are incorporated in and constitute a part of this specification, illustrate embodiments of the invention and, with a detailed description of the embodiments given below, serve to explain the principles of the invention.

FIG. 1 is a flow diagram of a typical wet milling ethanol production process.

FIG. 2 is a flow diagram of a typical dry milling ethanol production process.

FIG. 3 is a flow diagram of typical dry fraction ethanol production process.

FIGS. 4, 4A, and 4B are flow diagrams of systems for and methods of separating high value by-products from grains used for alcohol production in accordance with some embodiments of the present invention.

FIGS. 5 and 5A are flow diagrams of systems for and methods of separating high value by-products from grains used for alcohol production in accordance with some embodiments of the present invention.

FIGS. 6 and 6A are flow diagrams of systems for and methods of separating high value by-products from grains used for alcohol production in accordance with some embodiments of the present invention.

FIGS. 7, 7A, and 7B are flow diagrams of systems for and method of separating high value by-products from grains used for alcohol production in accordance with some embodiments of the present invention.

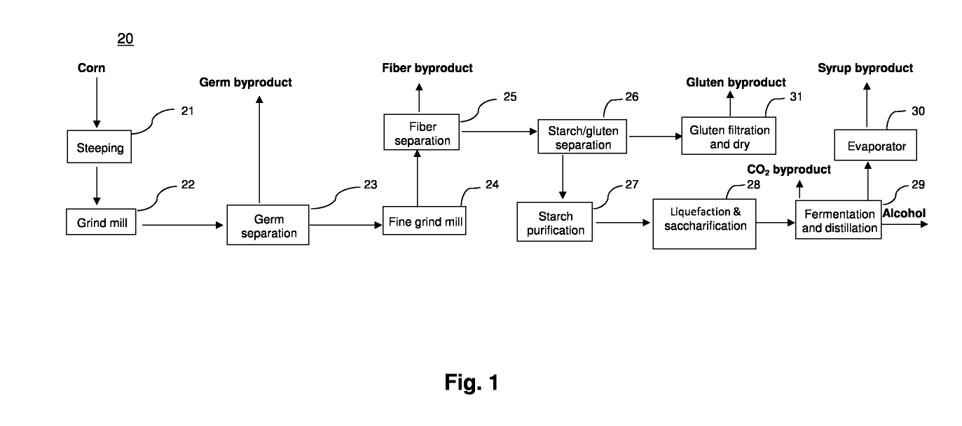

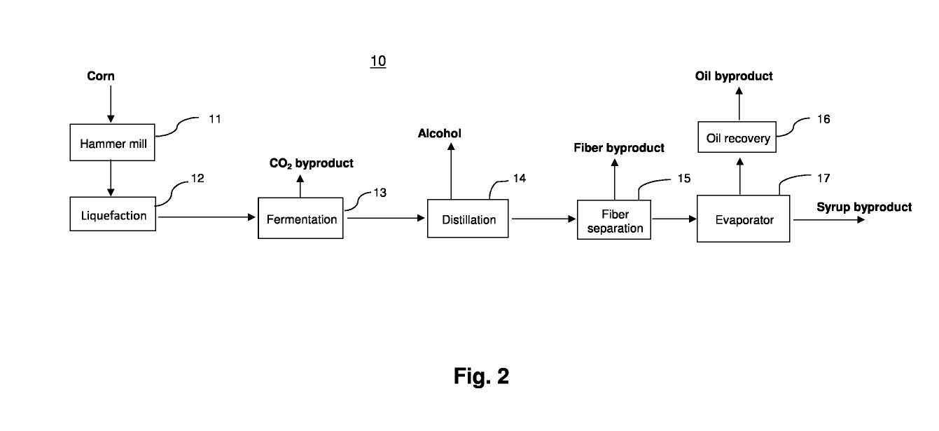

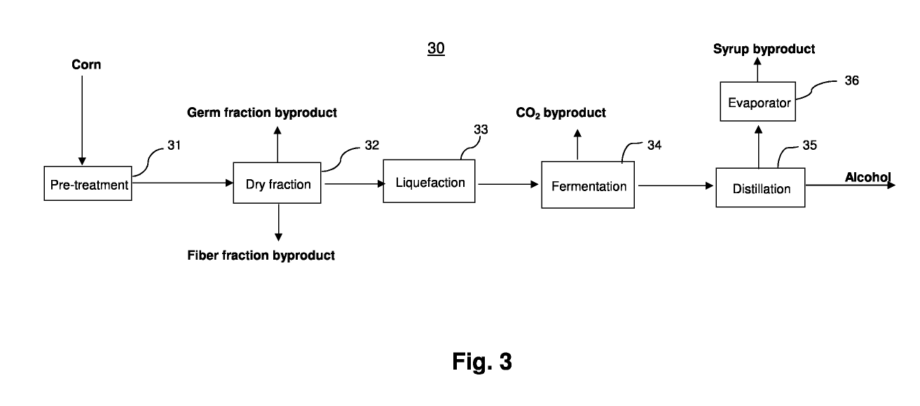

### DETAILED DESCRIPTION OF PREFERRED EMBODIMENTS

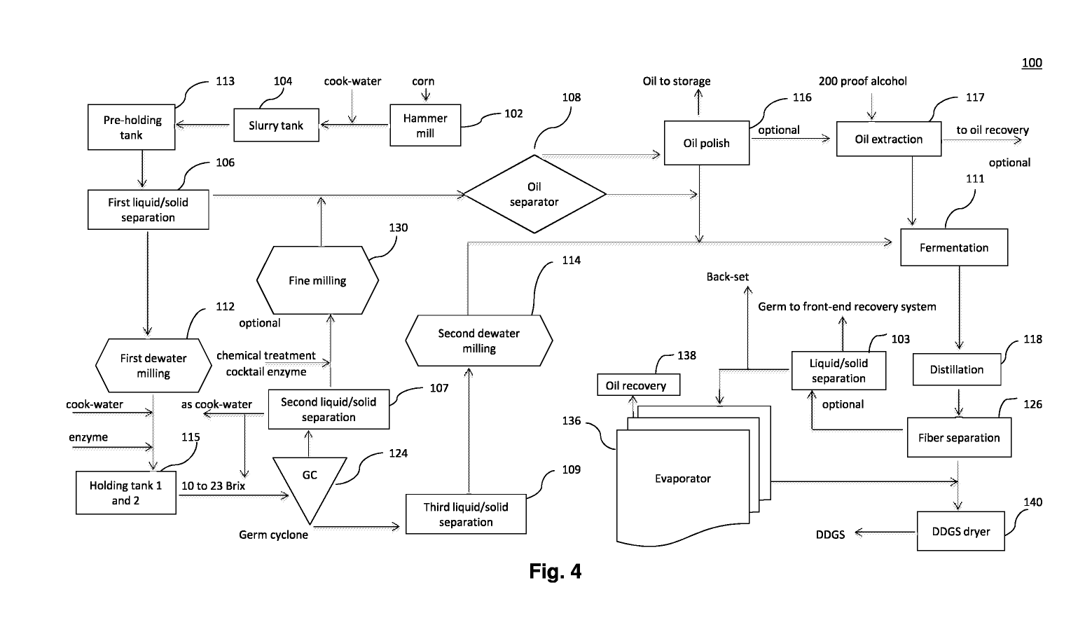

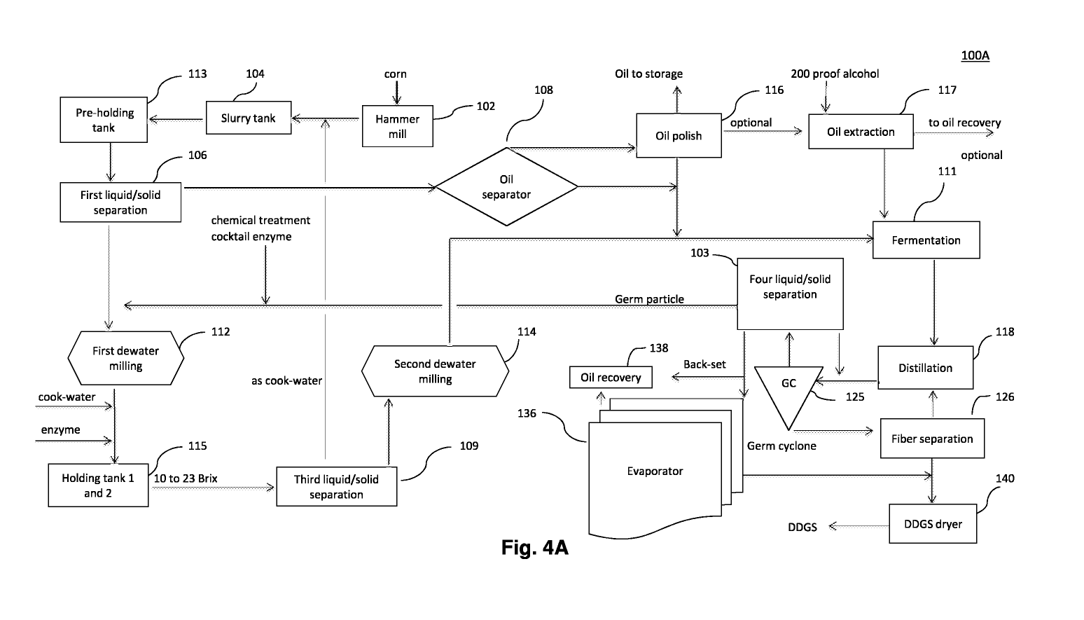

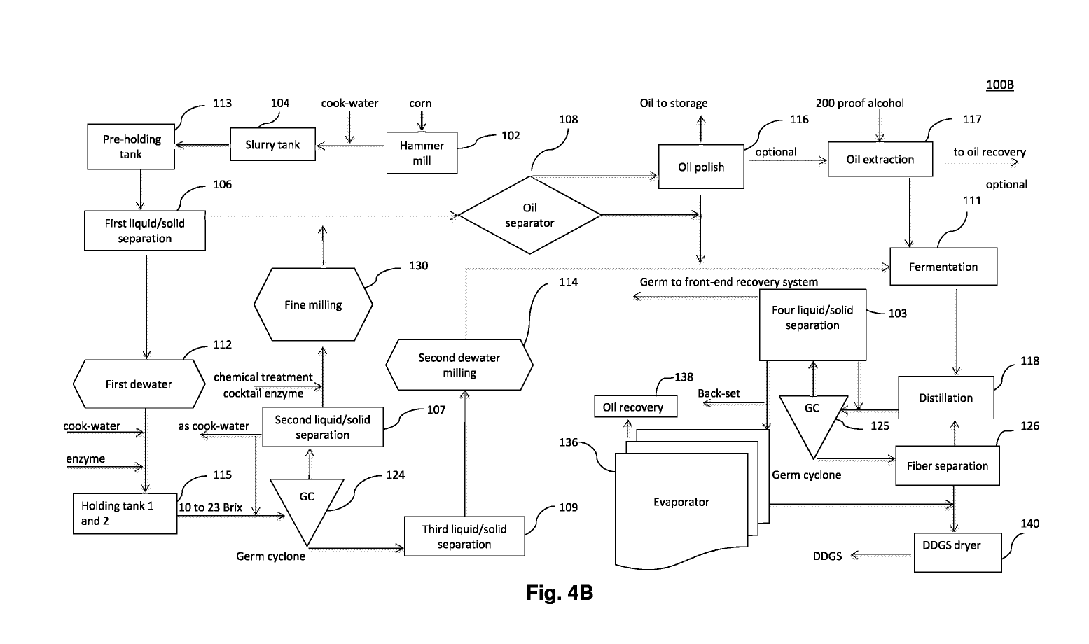

FIGS. 4-7B illustrate systems for and methods of separating high value by-products, such as oil and/or germ, from grains that are used for alcohol production in accordance with some embodiments of the present invention. These systems and methods are discussed in more detail herein below.

As an overview of some of the embodiments shown, each system and process described in the FIGS. 4-7B includes a germ separation system. The germ separation system is able to be used to separate germs from fiber by density differences in a heavy density liquid media. A similar process that uses density differences as the separation principle is described in PCT/US11/63228, which is incorporated by reference in its entirety for all purposes. The above mentioned germ separation system can be used in conjunction with one, two, or three front grinding mills in the front-end, the back-end, or both. A similar process that combines the germ separation system and one or more grinding mills is described in PCT/US11/63228, which is incorporated by reference in its entirety for all purposes. As described above, the front-end is defined as the process/steps that are performed before the fermentation process. The back-end is defined as the process/steps that are performed after the fermentation process.

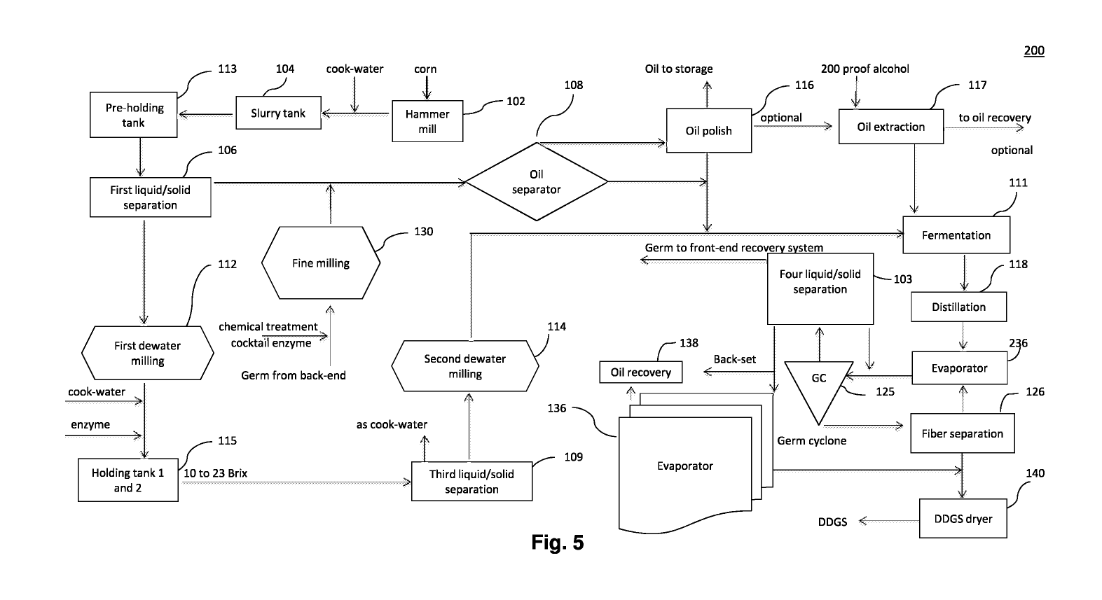

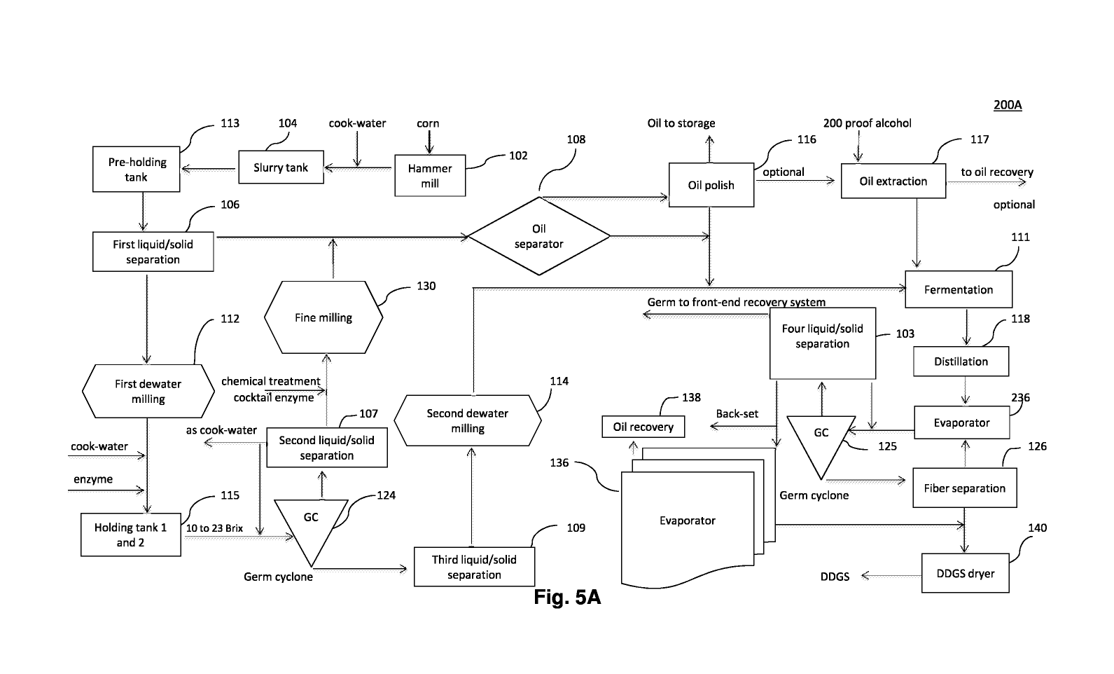

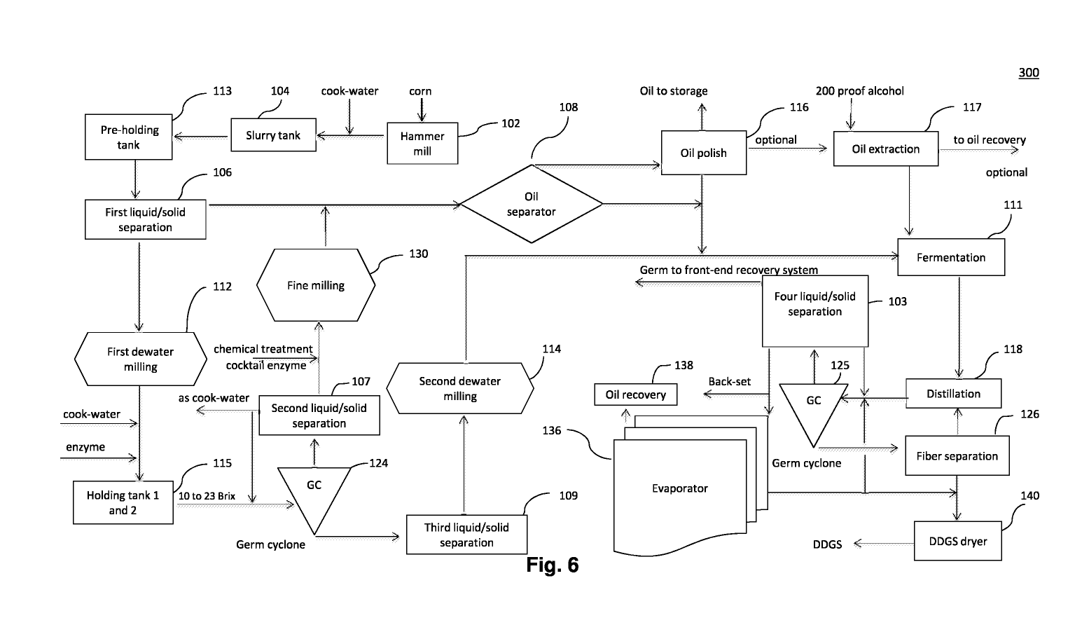

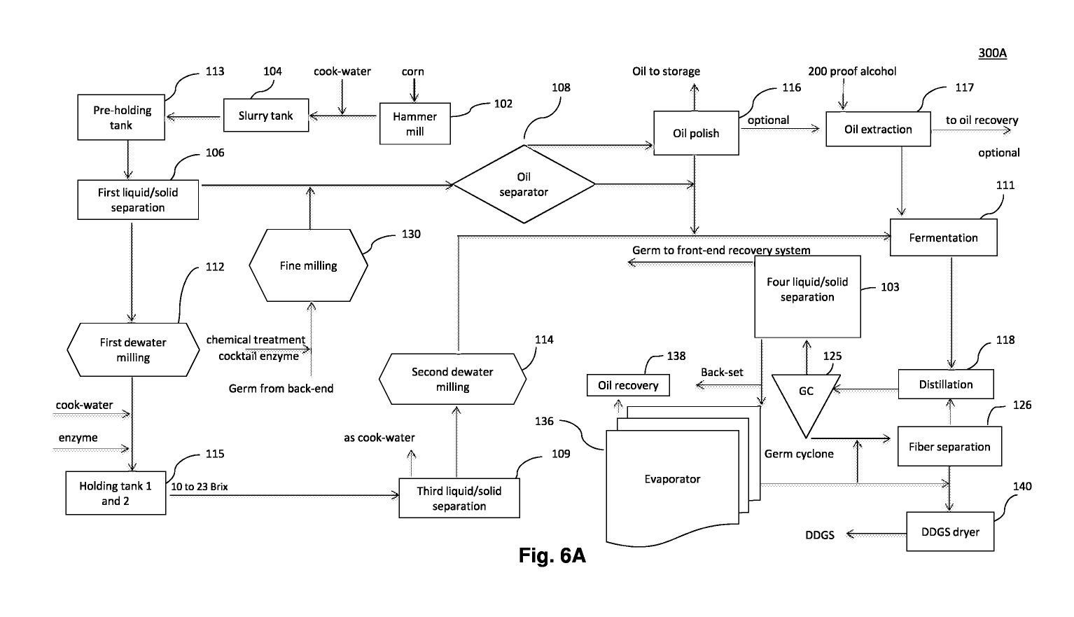

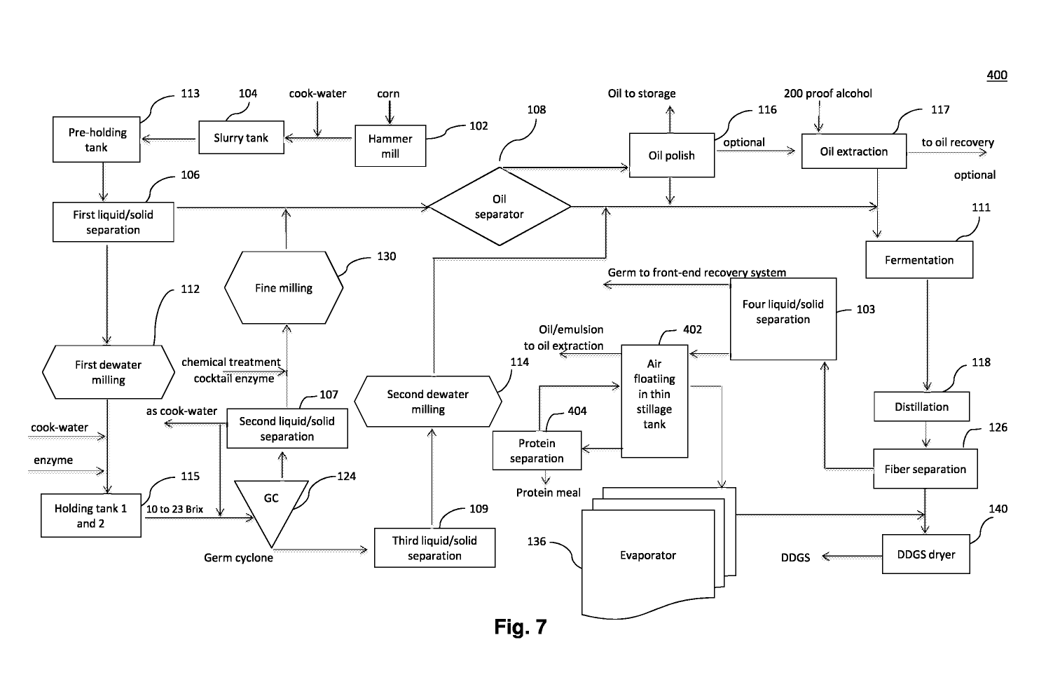

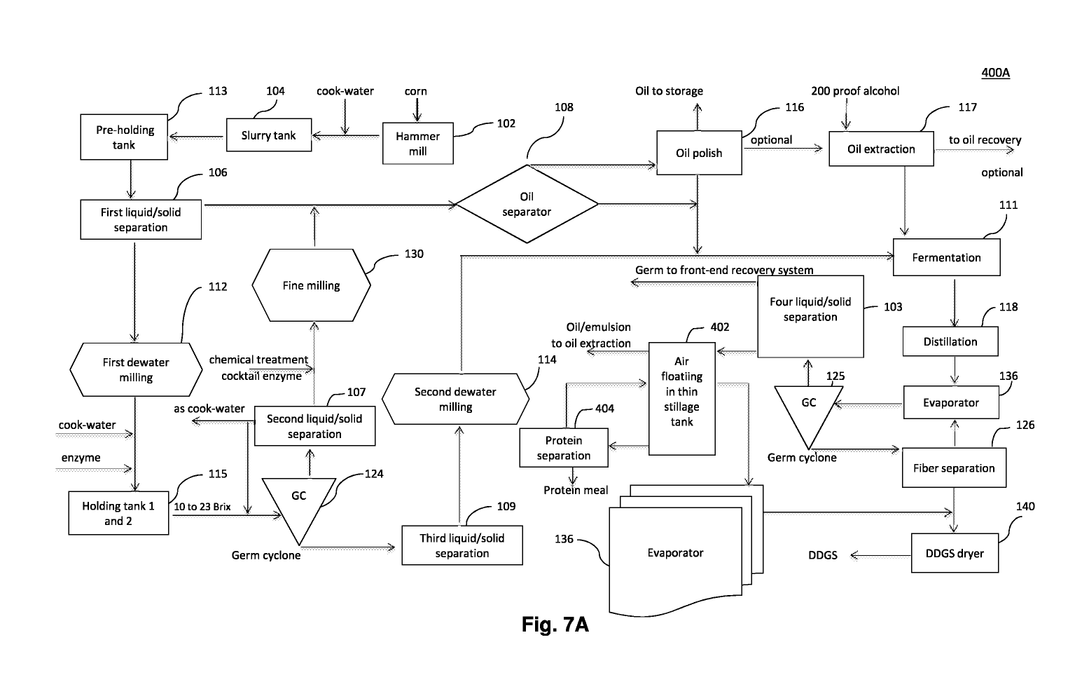

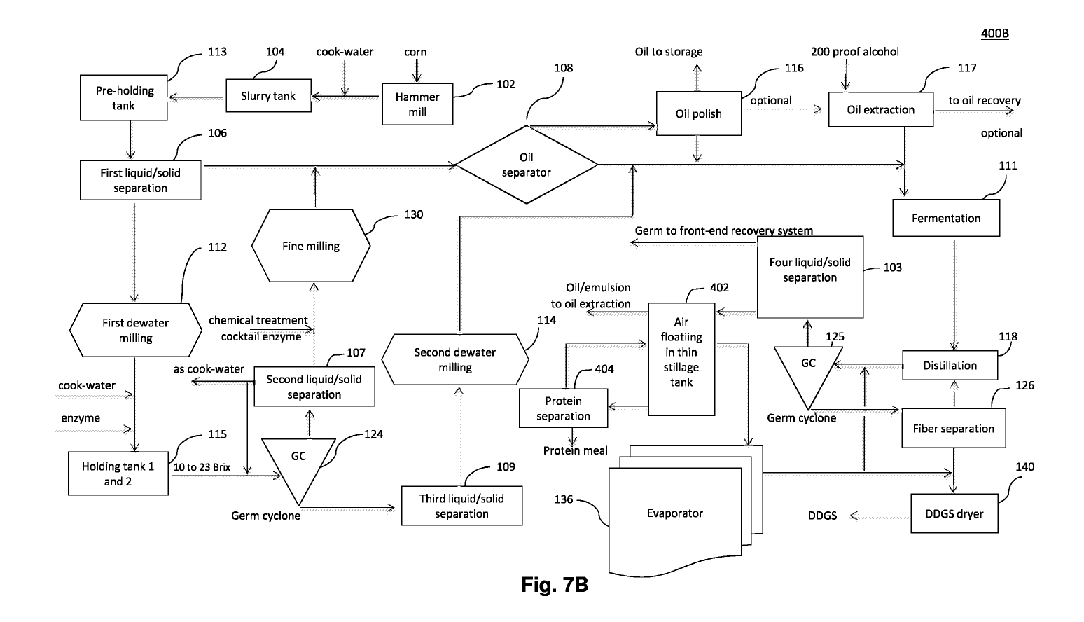

In a typical dry mill process, the viscosity of the liquefied starch in the front-end is about 24 to 35 Brix, which is too high for floating germs in the liquefied starch. In some embodiments of the present invention, the viscosity of the liquefied starch is adjusted/adjustable by using a counter current washing fluid/process. A similar process is described in the PCT US2012/030337, which is incorporated by reference in its entirety. The liquefied starch solution in the front-end has 2% to 29% Brix (density of 1.01 to 1.125) dependent on the amount of the cook-water (counter current washing liquid) that is added to the holding tank, so a desired/pre-defined density range of the heavy density liquid media in the front-end is able to be maintained in a range of 1.05 to 1.1 (12 to 23% Brix) as shown in the FIG. 4, FIG. 4A, and FIG. 4B. However, in the back-end, the whole stillage in a typical dry mill process contains less than 14% DS (density of 1.05). Having a low density, only a portion of the germ is able to be floated (the density of germ is normally in the range of 1 to 1.05 gram/ml). There are two ways to increase the density of the thin stillage for better germ separation including: a) feeding the whole stillage to the first effect evaporator so that the whole stillage is able to be concentrated to 15 to 23% DS as shown in the FIG. 5 and FIG. 5A and b) recycling a portion of the concentrated syrup from evaporator back to the whole stillage, so that the whole stillage has 15 to 23% DS with additional syrup, as shown in the FIG. 6 and FIG. 6A. The air floatation unit also can be added to improve the oil/protein recovery as shown in the FIG. 7, FIG. 7A, and FIG. 7B.

FIG. 4 and FIG. 7 illustrate systems with a front grinding mill and front germ separating systems. FIG. 7 has an additional air floatation system for oil and germ to recycle back to the front-end as part of the back-set stream. The FIGS. 4A, 5, and 6A are front grinding mills with a germ separating system in the back-end. In FIG. 4A, there is less than 14% of the DS in the whole stillage and less than 8% of the DS in thin stillage (density of 1.03, which can float only a portion of the germ). In FIGS. 5, 5A, and 7A, the density of liquid increases by preconcentrating the whole stillage to above 15% DS. In FIG. 6, FIG. 6A, and FIG. 7B, the density of the whole stillage is increased to above 15% DS by recycling a portion of the concentrated syrup from the evaporator. The systems in FIGS. 4B, 5A, 6, 7A, and 7B include a front grinding mill with two germ separation systems in both the front-end and the back-end to maximize the germ recovery.

In the FIG. 4B, the normal whole stillage has less than 14% of DS. In the FIG. 5A and the FIG. 7A, the whole stillage has 15% to 23% of DS by pre-concentrating the whole stillage at the first effect evaporator. The system in the FIG. 7A has an additional air floatation unit. FIGS. 6 and 7B with whole stillage have 15% to 23% of DS by recycling the syrup back. The FIG. 7B has an additional air floatation unit to float the oil/fine germ/protein layer by using air bubbles or by adding fine grinded germ slurry with Na₂CO₃ pre-treatment to substitute the air in the air floatation unit. The fine grinded germ slurry with Na₂CO₃ has a pH of 7.5 to 9, which is mixed with a thin stillage (a pH in the range of 4 to 5) to make a solution having a pH around 6. The fine CO₂ bubbles help to float the oil/fine germ/protein particles to the top of thin stillage tank. The oil/fine germ/protein particles are sent back to the front-end as part of back-set flow to recover the oil and protein. An enzyme, such as a cell wall degrading enzyme including glucoamylase, fungal, cellulose, cellubiose and protease, or any combinations thereof, can also be optionally added during the fine grinding Step 130 (FIGS. 4, 4B, 5, 5A, 6, 7, 7A, and 7B), for example, to help break cell wall in germ particles and release more oil from the germs.

Referring to FIG. 4, this figure is a flow diagram of a dry grinding ethanol production process and system 100 with a front-end milling procedure in accordance with some embodiments of the present invention for improving alcohol and/or byproduct yields (e.g., oil and/or protein yields). In the process 100, the front grinding process is described in PCT/US 2012/030337, which is incorporated by reference in its entirety for all purposes. Additional germ separation Steps 124 and 107 using heavy density liquid media to float and recover the germ and to be separated from fiber by density differences (the germ is lighter than the heavy density liquid media and the fiber is heavier than the heavy density liquid media) are included in the process 100.

At Step 102, corns are ground in a hammer mill with a screen size of 7/64 inch. At Step 104, the corn flour is mixed with cook-water to form slurry in a slurry tank. In some embodiments, the cook water is able to be the filtrate from the second/liquid separation Step 107 containing a 10 to 18 Brix liquefied starch solution to give about 30% of DS slurry. At Step 113, the slurry is fed to a jet cooker in a pre-holding tank to bring the temperature up to 260° □F. and hold for about 3 minutes at a U-tube, such that the slurry forms liquefied starch slurry. In some embodiments, an additional slurry tank is added with a stream injection to keep a temperature at 210° F. for more than a hour to replace the jet cooker. At Step 106, the liquefied starch slurry then feeds to a device for a solid/liquid separation process. The paddle screen with screen size in a range of 50 to 400 micron is used. A person of ordinary skill in the art will appreciate that any other screen type device is also able to be used. The filtrate at the Step 106 goes to the Step 108 and the solid portion goes to the Step 112.

At Step 108, the filtrate (containing mainly liquefied starch solution with oil, protein, fine fiber and fine germ particles with soluble solid in the corn kernel) from the Step 106 is fed to an oil separator to separate/recover the oil/emulsion/fine germ (light phase) from grit and fiber in liquefied slurry (heavy phase). A three phase nozzle centrifuge, or three phase decanter, or disc centrifuge can be used at the Step 108. At Step 116, the light phase discharged from the Step 108 goes to the oil polishing centrifuge to separate/recovery clean oil from the emulsion/germ particle layer. The clean oil discharge from the Step 116 (the oil polishing centrifuge) goes to an oil storage tank. At the Step 117, the emulsion/fine germ layer (as heavy phase discharge) discharged from oil at the Step 116 is able to be optionally mixed with 200 proof alcohol from distillation for an oil extraction step to extract more oil.

At a Step 112, the cake phase from the Step 106 (paddle screen) contains fiber, grit, germ in liquefied starch liquid having about 40% DS is received. At the Step 112, the cake from the Step 106 is fed to a first dewater milling Step 112. The first dewater milling step of the Step 112 is optimized to break the grit and germ particles as much as possible and to minimize the fiber particles from breaking At Step 115, the ground solid from the Step 112 is mixed with a portion of cook-water (the water from back-set and evaporator) and is held at one or more holding tanks at about 140 to 180° F. for a total of 3 to 5 hours holding time. In some embodiments, more than two holding tanks are used in series. Some amount of fresh Alpha Amylase with an amount of cell breaking enzymes, optionally, such as Glucoamylase, fugal, cellulose, cellobisoe and protease are used. By adding the enzymes, the germ particles and grit are further broken down and more oil is released. The starch continues to further liquefy in the two holding tanks at a lower Brix solution to soften germs, which is faster than the solution with 25 to 29 Brix in a typical system. At Step 124, the density of this slurry at the holding tank (Step 115) is adjusted by an amount of cook-water to have a density in the range of 1.05 to 1.1 gram/ml range (about 15 to 23 Brix), so that the germ particles float in this slurry and can be separated out from the fiber (the fiber is heavier than the density range and sink in the solution) at the germ separation step. A 6 to 9 inch germ cyclone (which is used in the corn wet milling process) is ideal as one of the embodiments for the germ separation step 124. In some embodiments, a 6 inch cyclone, such as a hydro-cyclone, is used. In some other embodiments, 8 and 9 inch germ cyclone are used. In other embodiments, a 9 inch germ cyclone followed by an 8 inch germ cyclone gives an optimized result. In some embodiments, two/three sets of cyclones in series recover more germs and give better separation. In some embodiments, germ cyclone followed by a decanter centrifuge is used. The decanter has a better germ/fiber separation result at a high concentration. However, equipment costs of using the decanter are much higher.

At Step 107, the overflow from the germ separator Step 124 containing germ particles, protein, and portion fine fiber in liquefied starch slurry is fed to a second solid/liquid separator. The paddle screen used at the Step 107 is able to have a screen opening size from 50 to 150 microns. The filtrate from paddle screen containing mainly liquefied starch solution (containing mainly protein with fine fiber) is sent back to the slurry tank (Step 104) as part of the cook-water (fresh cook-water is able to be added as needed). The portion of this filtrate can also be recycled back as part of a germ cyclone feed to dilute the slurry inside the germ cyclone, such that the germ recovery efficiency is able to be improved. At

Step 130, the cake from the second liquid/solid separation (Step 107) can be mixed with a cell breaking enzyme and/or be adjusted to have a pH between 5 to 8 before sending the cake to a fine grinding device to grind the germ particles to a size smaller than 150 microns. The grinding mill with a fine grinding plate or any high shear force mills are able to be used at the Step 130.

At Step 108, the ground germ slurry is sent from the fine milling (Step 130) to an oil separator for oil recovery. The cake from the second liquid/solid separation device at the Step 107 can also be optionally sent to the first dewatered grinding device at Step 112, such that the fine milling Step 130 is able to be not used in some embodiments. The overflow from a germ cyclone separating Step 124 can also be optionally sent to the slurry tank at the Step 104 directly (without going through the second solid/liquid separating device at Step 107 and the fine milling device at Step 130) as cook-water. The germ particles are able to be ground by the first dewater milling Step 112 to save the equipment costs.

At Step 109, the underflow from the germ separator at Step 124, containing mainly fiber and grit in heavy liquefied starch slurry, is sent to a third solid/liquid separator. The paddle screen or special design classification decanter can be used at the Step 109 to dewater and further remove germs from fiber. The filtrate from the paddle screen or overflow from the classification decanter at Step 109 is recycled back to the germ separator at Step 124.

At Step 114, the cake from the third solid/liquid separation Step 109 is optionally fed to a second dewater milling device to further break down the germ and grit particles and bonds among fiber, starch, protein, and oil. The grinding mill with devil tooth is ideal for the second dewater milling. At the fermenting Step 111, the ground solid from the second dewatered milling at Step 114 is mixed with the heavy phase and nozzle flow from the oil separator at the Step 108, heavy phase flow from oil polish centrifuge at the Step 116, and oil extraction at the Step 117.

At the fermenting Step 111, the liquefied starch in the above mentioned stream goes to a fermenter and the sugar contained in the liquefied starch is converted to alcohol. In some embodiments, the alcohol comprises ethanol, butanol, or a combination thereof. Other alcohols are within the scope of the present invention. Other additional procedures to process/synthesize other alcohols are able to be incorporated herein. For example, a carbon-carbon coupling reaction, such as a Suzuki coupling/reaction, is able to be used to synthesize long chain alcohols, such as coupling two ethanol molecules to become a butanol molecule.

At Step 118, a distilling step is performed to recover the alcohol at a distiller. The stream discharge from the bottom of the distiller at the Step 118 is a whole stillage, which contains about 13 to 14% of DS in a typical dry milling process. At Step 126, the whole stillage is fed to a fiber separating device to separate a fiber from protein and recover the fiber as DDG cake having about 35% of DS. A decanter centrifuge is able to be used at the Step 126.

At Step 103, the overflow from the decanter from the Step 126 contains around 8% of DS with a small amount of germ particles, which is able to be optionally sent to other liquid/solid separator to recover more germs. A portion of the overflow from the decanter at the Step 126 or from the liquid/solid separating Step 103 is able to be used as a back-set flow to be supplied to the holding tank at the Step 115. At Step 136, the filtrate from the liquid/solid separator at the Step 103 is sent to an evaporator. At Step 138, the back-end oil recovery system can also be optionally added to the evaporator to recover oil. At Step 140, the decanter cake and syrup (about 40% DS) are sent to DDGS dryer to produce DDGS as a byproduct. The dry grinding ethanol production process and system 100 is able to increase the oil production yield about 0.2 lb/Bu.

FIG. 4A is a flow diagram of the dry grinding ethanol production process and system 100A with a front-end milling method in accordance with some embodiments for improving alcohol and/or byproduct yields. The yields of the byproducts include oil and/or protein. The process 100A of FIG. 4A is a variation of the dry grinding ethanol production process 100 of FIG. 4. (In FIG. 4, the germ separating system 124 and the second liquid/solid separating Step 107 are at the front-end before the fermenting Step 111; whereas, in FIG. 4A, the germ separating system 125 and the liquid/solid separator 103 are located at the back-end.) In the process 100A of the FIG. 4A, the Step 102 (hammer milling) to Step 115 (holding tanks) is able to be the same as the steps described in FIG. 4. In FIG. 4A, the stream after two holding tanks at Step 115 goes to the Step 109 (the third liquid/solid separating step; without going through a germ separating step). The filtrate from Step 109 is sent to a slurry tank (Step 104), which is used as a portion of cook-water.

At Step 114, the solid from the Step 109 is sent to the second dewatering mill. At fermenting Step 111, the ground solid from the second dewatered milling at Step 114, the underflow from the oil separating Step 108, the underflow from the oil polishing centrifuging Step 116, and oil from the oil extracting Step 117 are mixed together and sent to a fermenter to convert sugar to alcohol.

At Step 118, distillation is performed after the fermenting Step 111 to recover alcohol. The whole stillage from the bottom of distillation tower of the Step 118 contains about 13% to 14% of DS with a density about 1.05 gram/ml, which is very close to the density of germ, 1 to 1.05 gram/ml.

At Step 125, the whole stillage from the Step 118 is sent to a germ separator, such that the germ is able to be separated out from the solution. A portion of the germ at the Step 125 (about 10 to 30% germ in feed) floats on top and is discharged with a light phase. About 70 to 90% of germ traps and settles with fiber and is discharged as the decanter cake at the fiber separation device/decanter at Step 126. The overflow from the germ separator at Step 125 is sent to a liquid/solid separator at Step 103 to recover the germs. The germ particles are treated by an enzyme or pH adjustment and are milled by a fine grinding mill to break the cell wall and release the oil from the germ particles. The oil released from the germs is recovered in the front-end at Steps 108, 116 and 117 or in the back-end of the oil recovery system at Step 138. A portion of the filtrate from the Step 103 is sent back to the germ cyclone feed tank as a back-set flow to dilute the feed and improve the germ recovery efficiency. Another portion of the filtrate is sent to an evaporator 136 to be concentrated to become a syrup. The process 100A of FIG. 4A is able to increase the oil yield by 0.05 lb/Bu.

FIG. 4B is a flow diagram illustrating a dry grinding ethanol production process and system 100B with a front-end milling method in accordance with some embodiments for improving alcohol and/or byproduct (oil and/or protein) yields. In the process 100B, two germ separation systems (Steps 124 and 107 at the front-end and Steps 125 and 103 at the back-end) are used. The system of FIG. 4B is a combination of the systems described in FIG. 4 and FIG. 4A. In some experiments, the oil yield increased using the system and process described in FIG. 4B is close to 0.25 lb/Bu.

FIG. 5 is a flow diagram illustrating a dry grinding ethanol production process and system 200 with a front-end milling method in accordance with some embodiments of the present invention for improving alcohol and/or byproducts (oil and/or protein) yields. The process 200 is a variation of the dry grinding ethanol production process 100. The process 200 has only one germ separation device/process (e.g., Steps 125 and 103 at the back-end of FIG. 4A) The whole stillage has 13% to 14% of DS with about 5% of soluble solid and a density around 1.05 gram/ml. The thin stillage contains about 8% of DS and has a density of 1.03 gram/ml.

In a typical process, the density of the germ particles is very close to the density of the slurry, so the germ particles trap and settle with fiber and discharge as part of decanter cake at a fiber separation step. In contrast, in some embodiments of the present invention, the density of the whole stillage is increased by feeding the whole stillage to an evaporator at Step 236 before the whole stillage is sent to the germ separator at Step 125. At the Step 236, the density of the whole stillage is increased to about 15% to 23% of DS before the whole stillage is fed to a germ separator at Step 125. By increasing the whole stillage density to about 1.05 to 1.1 gram/ml, more germs go to the decanter overflow, such that more oil is able to be recovered. The rest of the process is able to be the same as the process described in FIG. 4A. The oil yield using the process described in FIG. 5 is able to increase around 0.15 lb./Bu. Additional germ separation steps, such as Step 124 and Step 107 described in FIG. 5A, at the front-end process can be added to the process of FIG. 5.

FIG. 5A is a flow diagram illustrating a dry grinding ethanol production process and system 200 with a front-end milling device in accordance with some embodiments of the present invention for improving alcohol and/or byproducts (oil and/or protein) yields. The process 200A described in FIG. 5A is a variation of the dry grinding ethanol production process 100. The process 200A has two germ separating steps/devices (both at the front-end and at the back-end). The difference between FIG. 4B and FIG. 5A is that the whole stillage is pre-concentrated to 15% to 23% DS in FIG. 5A, so the germ separating Step 115 is able to float more germs than the procedure of FIG. 4B. The oil yield using the process described in FIG. 5A is able to increase about 0.3 lb./Bu.

FIG. 6 is a flow diagram illustrating a dry grinding ethanol production process and system 300 with a front-end milling method/device in accordance with some embodiments of the present invention for improving alcohol and/or byproduct (oil and/or protein) yields. The process 300 is a variation of the dry grinding ethanol production process 200A. The process 300 has a front-end milling method/device. In FIG. 6, the density of the whole stillage is increased by adding/recycling a portion of the heavy syrup from an evaporating Step 136 to the germ separating Step 125, which is different from the pre-concentrating the slurry by a first effect evaporator as described in the FIG. 5A. The process in FIG. 6 increases the oil yield about 0.3 lb./Bu.

FIG. 6A is a flow diagram illustrating a dry grinding ethanol production process and system 300 with a front-end milling method in accordance with some embodiments of the present invention for improving alcohol and/or byproduct (oil and/or protein) yields. The process 300 is a variation of the dry grinding ethanol production process 200. The process 300 has a front-end milling device. In FIG. 6A, the density of the whole stillage is increased by adding/recycling a portion of heavy syrup to the whole stillage, which is different from pre-concentrating in the first effect evaporator as described in the FIG. 5. By using the process of FIG. 6A, oil yield increases around 0.15 lb/Bu.

FIG. 7 is a flow diagram illustrating a dry grinding ethanol production process and system 400 with a front-end milling method in accordance with some embodiments of the present invention for improving alcohol and/or byproducts (oil and/or protein) yields. The process 400 is a variation of the dry grinding ethanol production process 100. The process 400 has a germ recovering system (e.g., germ separating Step 124 and liquid/solid separating Step 107). The process in FIG. 7 includes an air floating Step 402 to the current thin stillage tank. A protein separating Step 404 is able to be added to as an optional feature. The air floating Step 402 carries the oil/emulsion and fine germ particles by small air bubbles in the thin stillage tank. The light layer (oil/emulsion/fine germ on top of thin stillage tank) is collected on the top of the thin stillage tank and is sent to a front-end step, such that more oil from the oil recovering Step 108, oil polishing Step 116, and oil extracting Step 117 is able to be recovered. In some embodiments, the air floating Step 402 is able to be performed by pre-treating the fine ground germ particle slurry by adding an amount of Na₂CO₃ to the thin stillage tank. The fine ground germ slurry having a pH of 7 to 9 can partially neutralize the acid in the thin stillage and cause a lot of fine CO₂ bubbles to be formed. (The pH of the thin stillage is around 3-5 before adding the Na₂CO₃) The fine CO₂ bubbles with the increased pH in the thin stillage speed up the floatation and facilitate more oil/protein from syrup to be recovered. The solid at the bottom of the thin stillage tank (mainly protein and fine fiber) can be optionally recovered from the bottom of the thin stillage tank by the protein separating Step 404 to produce 50% of the protein meal. A high speed decanter or a disc decanter can be used in the above described process. The oil yield increase is around 0.4 lb./Bu.

FIG. 7A is a flow diagram illustrating a dry grinding ethanol production process and system 400A with a front-end milling method in accordance with some embodiments of the present invention for improving alcohol and/or byproduct (oil and/or protein) yields. The process 400A is a variation of the dry grinding ethanol production process 200A with a front-end milling method described in FIG. 5A. In the FIG. 7A, an air floatation system is added to the process described in the FIG. 5A. The air floating Step 402 carries the oil/emulsion and fine germ particles by using small air bubbles at the thin stillage tank. The air floating Step 402 is able to be performed using the process described in the FIG. 6A. The light layer (oil/emulsion/fine germ on top of the thin stillage tank) can be taken out with a back-set flow and to be recycled back to the front-end, such that more oil is able to be recovered from the oil recovering Step 108, the oil polishing Step 116, and the oil extracting step 117. In some embodiments, the back-end oil recovery system described in the FIG. 5A can be eliminated in the process described in the FIG. 7A. In some embodiments, the air floating Step 402 is able to be performed by pretreating the fine ground germ particle slurry by adding Na₂CO₃ to the thin stillage tank. The fine ground germ slurry having a pH of 7 to 9 can partially neutralize the acid in the thin stillage and cause many fine CO₂ bubbles to be formed. (the pH of the thin stillage is around 3-5 before adding the Na₂CO₃) The fine CO₂ bubbles and the increased pH in the thin stillage are able to speed up the floatation and facilitate more oil/protein from syrup to be recovered. The solid at the bottom of the thin stillage tank, mainly protein and fine fiber, can be optionally recovered from the bottom of the thin stillage tank by the protein separating Step 404 to produce 50% of the protein meal. A high speed decanter or a disc decanter can be used in the above described process. The process described in the FIG. 7A can be another variation of the process described in the FIG. 7 by adding the pre-concentrating Step 136 to increase the thin stillage density. Using the process in FIG. 7A, the oil yield increases around 0.45 lb./Bu.

FIG. 7B is a flow diagram illustrating a dry grinding ethanol production process and system 400B with a front-end milling method in accordance with some embodiments for improving alcohol and/or byproduct (oil and/or protein) yields. The process 400B is a variation of the dry grinding ethanol production process 300 with a front-end milling method of FIG. 6. In the FIG. 7B, an air floatation system is added to the FIG. 6. The air floatation Step 402 carries the oil/emulsion and fine germ particles by small air bubbles in the thin stillage tank. The light layer (oil/emulsion/fine germs are on top of thin stillage tank) can be taken out by the back-set flow and can be recycled back to the front-end, such that more oil is able to be recovered by the oil recovering Step 108, the oil polishing Step 116, and the oil extracting Step 117. In some embodiments, the back-end oil recovery system described in FIG. 6 can be eliminated in the process described in the FIG. 7B. In some embodiments, the air floatation Step 402 is able to be performed by pretreating the fine grinded germ particle slurry through adding Na₂CO₃ to thin stillage tank. The fine ground germ slurry having a pH of 7 to 9 can partially neutralize the acid in the thin stillage and cause many fine CO₂ bubbles to be formed. (the pH of the thin stillage is around 3-5 before adding the Na₂CO₃) The fine CO₂ bubbles and the increased pH in the thin stillage are able to speed up the floatation and facilitate more oil/protein from syrup to be recovered. The solid at the bottom of the thin stillage tank, mainly protein and fine fiber, can be optionally recovered from the bottom of the thin stillage tank by the protein separation Step 404 to produce 50% of the protein meal. A high speed decanter or a disc decanter can be used in the above described process. The FIG. 7B can also be another variation of FIG. 7 by recycling the syrup to increase the thin stillage density. By using the process in FIG. 7B, the oil yield increases around 0.45 lb./Bu.

While the present invention has been illustrated by a description of various embodiments and while these embodiments have been described in considerable detail, it is not the intention of the applicant to restrict or in any way limit the scope of the appended claims to such detail. For example, although the various systems and methods described herein is about the process on corns, virtually any other type of grains, including, but not limited to, wheat, barley, sorghum, rye, rice, oats and the like, can be used. Also, for example, for the optional oil separating Step 108, the feed may be taken from the slurry tank 104, pre-holding tank Steps 113, or from the first or second holding tank Step 115. Also, for example, the front dewatering grinding step can be one or more steps in series. A person of ordinary skill in the art appreciates that the flow diagrams can be modified, for example, to include or exclude the fine grinding mills, the front-end and the back-end oil recovery system, the germ separation system in the front-end or in the back-end, and the protein separating steps; to vary the locations of the dewatering milling step(s), and to separate/recover germs by a heavy density liquid media; such that more oil from the germs are able to be extracted. Additional advantages and modifications will readily appear to those skilled in the art. The process described herein is able to used to/modified to produce other chemicals, such as butanol. The process can include liquefying the starch in grain to become sugar solution, separating the oil, protein, and fiber from the sugar solution, and converting the sugar solution to butanol, citric acid, and lysine. The term cyclone(s) used in this patent application includes hydro-cyclone(s).

Thus, the invention is not limited to the specific details, representative, apparatus and method, and illustrative example shown and described. Accordingly, variations are able to be made from such details without departing from the spirit or scope of applicant's general inventive concept/scope.

What is claimed is:

1. A method of separating germs from a whole stillage comprising increasing a liquid density of a higher density layer of a slurry after a distilling step.

2. The method of claim 1, wherein the increasing the liquid density comprises making the higher density layer having a solid content in the range of 15%-30% DS.

3. The method of claim 1, wherein the increasing the liquid density comprises optimizing the higher density layer having a solid content in the range of 17%-24% DS.

4. The method of claim 1, wherein the increasing the liquid density comprises sending the whole stillage to an evaporator before separating germs.

5. The method of claim 1, wherein the increasing the liquid density comprises adding a concentrated syrup to the whole stillage.

6. The method of claim 1, wherein the slurry comprises the whole stillage.

7. The method of claim 1, further comprising separating germs from fiber using a germ cyclone, a decanter or a combination thereof.

8. The method of claim 7, wherein the germ cyclone, the decanter or both comprises a single or a multiple stage separating device.

9. The method of claim 7, wherein the germ cyclone, the decanter or both comprises a counter current setup.

10. The method of claim 1, further comprising recovering germs from a thin stillage layer.

11. The method of claim 10, further comprising releasing oil by dewater milling the germs.

12. The method of claim 10, further comprising breaking a germ cell wall by the dewater milling.

13. The method of claim 10, further comprising breaking a germ cell wall by adding a cell breaking enzyme.

14. The method of claim 10, further comprising adjusting a pH value to be in the range of 6 to 9 before dewater milling.

15. The method of claim 10, further comprising recycling a portion of a de-germ thin stillage solution to dilute a feed to a germ cyclone such that a germ and fiber separating efficiency is improved.

16. The method of claim 10, further comprising floating fine germ particles, oil emulsion, or both by an air flotation system.

17. An alcohol production system comprising a first germ cyclone, a first decanter, or a combination thereof after a distiller configured to separate germs from fiber.

18. The system of claim 17, further comprising a second germ cyclone, a second decanter, or a combination thereof before the fermenter.

19. The system of claim 17, further comprising an evaporator before the first germ cyclone, the first decanter, or the combination thereof.

20. The system of claim 17, further comprising a distillation device before the first germ cyclone, the first decanter, or the combination thereof.

21. The system of claim 17, further comprising a dewater milling device before the fermenter.

22. The system of claim 17, further comprising a protein recovery device.

23. The system of claim 17, further comprising an oil recovering device before the fermenter.

24. The system of claim 17, further comprising an air floating device for recovering the germs in a thin stillage.

25. A method of recovering germs comprising reducing a viscosity of a liquefied starch solution.

26. The method of claim 25, wherein the liquefied starch solution has a reduced viscosity in the range of 12% and 23% Brix.

27. The method of claim 25, wherein the liquefied starch solution has a density in the range of 1.01 to 1.125.

28. The method of claim 25, wherein the reducing the viscosity is done by adding a cook-water.

29. The method of claim 25, wherein the cook-water comprises a counter current after separating germs from fiber.

30. The method of claim 25, wherein the reducing the viscosity occurs before or at separating germs from fiber.

31. A method of recovering germs from a liquefied starch solution comprising separating germs from fiber before fermenting using a germ cyclone, a decanter, or a combination thereof.

32. The method of claim 31 further comprising recovering the germs from the liquefied starch solution.

33. The method of claim 31 further comprising breaking cell walls of the germs to release oil by dewater milling.

34. The method of claim 33 further comprising adjusting a pH value in a range of 6 to 9 before the dewater milling.

35. The method of claim 31 further comprising breaking cell walls of the germs by adding an enzyme.

36. The method of claim 31 further comprising recycling a portion of a de-germ liquefied starch solution to dilute a feed to the germ cyclone, the decanter, or the combination thereof.

37. The method of claim 31, wherein the germ cyclone, the decanter, or the combination thereof comprises a single stage setup.

38. The method of claim 31, wherein the germ cyclone, the decanter, or the combination thereof comprises a multiple stage setup.

39. The method of claim 31, wherein the germ cyclone, the decanter, or the combination thereof comprises a counter current setup.

40. The method of claim 31, wherein the liquefied starch solution has a viscosity in a range of 8 to 35 Brix.

41. The method of claim 31, further comprising adjusting a viscosity of the liquefied starch solution by using a counter current washing process.

42. The method of claim 31 further comprising dewater milling before the fermenting.

43. The method of claim 31 further comprising recovering protein.

44. The method of claim 31 further comprising recovering oil before the fermenting.
